(19) United States
(12) Patent Application Publication
(10) Pub. No.: US 2023/0404964 A1
(43) Pub. Date: Dec. 21, 2023

(54) COMBINATION FOR REDUCING OXIDATIVE STRESS IN THE BODY AND MAINTAINING LIVER FUNCTIONS

(71) Applicant: Aleksandr Vladimirovich DIKOVSKIY, Limassol, Cyprus (CY)

(72) Inventors: Anna Aleksandrovna NIKITINA, Moscow (RU); Ekaterina Sergeevna PRYAHINA, Bugulma (RU); Artemii Aleksandrovich SERGEEV, Moscow (RU); Sergei Aleksandrovich TOLMACHEV, Khotkovo (RU); Vera Gennadievna CHANKINA, Novomoskovsk (RU)

(21) Appl. No.: 18/253,220

(22) PCT Filed: Nov. 16, 2021
(86) PCT No.: PCT/RU2021/000507

§ 371 Date: May 17, 2023

(30) Foreign Application Priority Data
Nov. 17, 2020 (RU) 2020137655

(51) Int. Cl. A61K 31/352; A61K 36/00; A61K 31/51; A61K 31/198; A23L 33/10; A61P 1/16

(52) U.S. Cl. CPC A61K 31/352; A61K 36/00; A61K 31/51; A61K 31/198; A23L 33/10; A61P 1/16

(57) ABSTRACT

The invention relates to a composition based on a natural lignocellulose complex, a source of dietary fiber exhibiting antioxidant and adsorption properties and dihydromyrecitin or quercetin, which has hepatoprotective activity when used together due to multidirectional action. This combination can be used to reduce oxidative stress and its inducers in the blood and organs, as well as maintain the detoxification function of the liver and its restoration, contains dihydromyricetin or quercetin in an amount of 5 to 1000 mg and hydrolysed lignin in an amount of 50 mg to 5000 mg. Technical result: the combination has an unexpected synergistic effect aimed at reducing oxidative stress and its inducers, manifested in a decreased level of malondialdehyde and acetaldehyde in the blood and organs, as well as in a beneficial effect on liver function.

The invention relates to medicine and focuses on a combination suitable for reducing oxidative stress and its inducers in the human body, namely in the blood and organs, as well as maintaining the detoxification function of the liver and its recovery, containing dihydromyricetin or quercetin and hydrolytic lignin in pharmaceutically effective amounts providing a therapeutically effective daily dose of the composition.

The combination is used for maintaining the detoxification function and restore the liver, including with alcohol and/or drug poisoning.

A decrease in oxidative stress and its inducers in the blood is due to decreased levels of malondialdehyde and acetaldehyde in the blood and in human organs.

Thus, the present invention also relates to using this composition for prevention and decrease of the intensity of diseases and conditions associated with increased levels of malondialdehyde (MDA) and/or acetaldehyde (AA) in the blood. Increased MDA and AA levels are typical for the diseases such as diabetes mellitus, cancer and infectious diseases, strokes, coronary heart disease; kidney, liver, and pancreas diseases. The combination can be used for maintaining the detoxification function and restoring the liver in different destructive conditions such as alcohol and drug poisoning.

Reducing oxidative stress, maintaining the detoxification function and restoring the liver are due to decreased inducers of oxidative stress in the blood.

The invention can be embodied as a biologically active food supplement that contains the mentioned composition and various additives.

The invention can also be embodied as a product for an effective therapeutic and prophylactic dietary nutrition that contains the said composition and relevant additives. It can be used for reducing oxidative stress and its inducers in the blood and organs, as well as maintaining the detoxification function and restoring the liver.

### PRIOR ART

There is an patent RU 2279278 C2 of Jul. 10, 2006, which discloses detoxification agents based on hydrolysed lignin and lactose for use in acute poisoning with drugs and alcohol. In particular, the known drug is used for treatment of the different exogenous and endogenous intoxications.

RU 2310463 C1 of Nov. 20, 2007, focuses on using a hepatoprotective probiotic that contains plant extract and bacteria on a carrier selected, for instance, from hydrolysed lignin to protect the liver in oxidative stress. A probiotic with a hepatoprotective effect and preparations based on it normalize the functions and metabolic processes in the liver, as well as contribute to normalisation of the biocenosis of the gastrointestinal tract.

Patent application CN 103355713 A of Oct. 23, 2013 (abstract) relates to an instant drink capable of eliminating the effects of alcohol and nourishing the stomach, which contains dihydromyricetin along with other components such as tomato juice, kudzu root extract, daidzen, vitamin B, citric acid, xylitol, aspartame and water in a defined proportion. The drink, which can reduce the effects of alcohol and nourish the stomach, can be used to reduce acetaldehyde-induced damage to the liver, brain and central nervous system, eliminate headaches, skin allergies, and long-term discomfort after alcohol intoxication.

The compositions known from the prior art did not solve the problem associated with reducing the level of oxidative stress and its inducers in the blood and human organs and maintaining the detoxification functions of the liver.

The effect of oxidative stress on human health, as well as the development of pathological conditions, finds more and more evidence in clinical practice. In this regard, markers of oxidative stress and some inducers of oxidative stress, as well as markers that characterize the antioxidant capacity of human blood, are becoming diagnostic and prognostic indicators in clinical practice.

Malondialdehyde (MDA) is a recognized marker of oxidative stress and lipid peroxidation (LPO) in the body. MDA is able to interact with DNA, proteins, lipoproteins and phospholipids, disrupting their normal activity. MDA is used as a prognostic indicator for coronary heart disease (CHD), myocardial infarction (MI), and angina pectoris. In addition to these diseases, increased MDA levels are characteristic of some infectious diseases, diabetes mellitus (DM), kidney diseases, severe forms of psoriasis, multiple sclerosis, oncological diseases, pancreatitis, strokes, etc.

Exposure to external toxicants, including alcohol, certain drugs, as well as microbial agents and their toxins, leads to an increase in MDA levels.

Infectious diseases, including HIV, intestinal toxicoinfections, are often accompanied by an increased MDA level [Z. A. Kambachokova, 2005, 10.3109/00365548809032453]. Chronic hepatitis is also accompanied by an increased MDA level.

Alcohol intake also increases blood MDA, indicating that ethanol intake causes significant oxidative stress in the human body.

Acute and chronic noncommunicable diseases are often accompanied by increased MDA levels. An increased MDA level (+50-+216%) was found in patients with chronic pancreatitis during an exacerbation of moderate severity (Merinova, Kozlova, Kolesnichenko et al., 2013).

A clear relationship between an increased blood MDA and the development of obesity, hyperlipidemia and hypercholesterolemia and the blood MDA has been demonstrated in a number of clinical studies. Oxidative stress was proven to be an additional pathogenetic factor that aggravates the course of dyslipidemic conditions, including a direct correlation between the MDA level and the atherosclerotic index.

Elevated MDA levels are typical for diabetes mellitus. They are increased against the background of dyslipidemia and the development of diabetic renal complications.

The relationship between the elevated MDA levels and various cardiovascular diseases was revealed. In patients with chronic heart failure, blood MDA was significantly increased (2.65±1.03 mmol/1) compared to healthy people of the same age (1.45±0.77 mmol/1). The dependence between the MDA level and the severity of chronic heart failure has been shown in clinical studies. The predictive value of the MDA level for prediction of the survival of patients with severe cerebral artery infarction has been shown in clinical studies. MDA values >2.27 nmol/ml were found to directly correlate with 30-day mortality in patients.

High MDA levels were observed in patients with chronic renal failure (CRF), including those who need dialysis (3.98±0.6 nmol/ml). However, the dialysis procedure was found to increase blood MDA, especially with cellulose membranes used for dialysis. Another study involving patients with CKD stages 4 and 5 has shown that blood MDA is the most significant prognostic indicator for the development of metabolic syndrome, which significantly enhances the CKD severity and reduces the treatment efficacy.

High MDA levels have been found in patients with liver cirrhosis. The correlation of MDA level with the nervous system disorders was shown.

In patients with non-alcoholic fatty liver disease, high MDA levels were found −3.5±0.6 μm/l, 2.36-6.18 ng/ml, 3.03±0.23 nmol/ml.

An increased MDA level is also observed in the elderly, indicating decreased body defences against oxidative stress and an increased risk of developing age-related diseases.

Acetaldehyde (AA) is a toxicant, the main product of ethanol metabolism in humans and animals, one of the most powerful inducers of oxidative stress in humans. It is formed in the ethanol cleavage reaction, which is carried out by the enzyme alcohol dehydrogenase. AA accumulates in the liver, saliva, gastric acid, and the contents of the large intestine. Apart from the body's own cells that produce acetaldehyde after alcohol intake, acetaldehyde can be produced by microorganisms that live in the intestines and in the oral cavity, and these microorganisms form AA without the intake of exogenous ethanol. In addition, the intake of certain drugs, primarily proton pump blockers, promotes the production of AA, thus creating more favorable conditions for the development of certain bacteria types capable of synthesizing AA as a by-product of metabolism. In addition to being an inducer of oxidative stress, AA has a pronounced proliferative and mutagenic activity and oncogenic and toxic effects. The AA concentration significantly increases in smokers, because AA is one of the constituents of tobacco smoke.

The AA accumulation has been clinically proven to be one of the liver damage factors, in particular, in alcoholic liver damage. AA activates collagen synthesis, the expression of genes that encodes the synthesis of procollagen, fibronectin, growth factors, leading to the development of fibrotic pathological changes in the liver structure that arise mainly against the background of regular alcohol consumption.

Numerous studies have shown that an increased AA concentration directly or indirectly enhances the risk of developing bowel cancer, upper gastrointestinal tract cancer, pharynx and oral cavity cancer.

Experiments on rats have also proven that acute liver injuries are also accompanied by a significant increase in AA levels.

Thus, the purpose of this invention was to create a new composition for reducing oxidative stress, the level of inducers of oxidative stress in the blood and human organs, and maintaining liver functions, which contains a combination of lignin and dihydromyricetin or quercetin and has high stability, without limitations on use in all age groups and specific groups of patients due to the synergistic effect of active components found in the composition in effective amounts. The composition can be used as an oral dosage form, and can also be included in the composition of a biologically active food supplement, as well as in the composition of medical or preventive dietary food products.

One of the components included in the claimed composition is lignin, in particular, hydrolytic lignin, which is a natural polymer forming part of plant cells and exhibiting its unique adsorption and antioxidant properties.

In one embodiment of the invention, hydrolytic lignin can represent a natural lignocellulose complex containing up to 99 wt % of lignin and 1 wt % of cellulose.

The properties of lignin are preconditioned by the presence of a developed porous structure dominated by pores with a radius of more than 50 nm (up to 90% of the total porometric volume). However, the effective pore radii found in the preparation are in the range of 100-1000 nm, which characterizes lignin as a macroporous sorbent. The variety of polar and non-polar functional groups increases the sorption activity of lignin. The porous structure and functional groups are responsible for the ability of lignin to adsorb hydrophilic and hydrophobic compounds, as well as microorganisms, using the chemisorption, adsorption, and absorption [H. A. [symbol] [symbol] 1991] mechanisms. Lignin and preparations based on it have also shown the ability to sorb organic molecules having a protein and lipopolysaccharide nature, which allows it to be used for the sorption of microbial toxins and toxic substances of endogenous origin, molecules of average weight, allergens and xenobiotics[Leskinen T. et al. Adsorption of Proteins on Colloidal Lignin Particles for Advanced Biomaterials.//Biomacromolecules. 2017. Vol. 18, [symbol] . 9. p. 2767-2776]. [symbol] [symbol] [symbol] [symbol] [symbol] [symbol] [symbol] roc. [symbol] [symbol] [symbol] , 2007][ [symbol] [symbol] [symbol] [symbol] [symbol] [symbol] [symbol] [symbol] [symbol] [symbol] [symbol] // [symbol] [symbol] [symbol] . 2003. Vol. 37, [symbol] 5. P. 28-32].

Another active component of the combination is dihydromyricetin or quercetin.

Dihydromyricetin (DHM) is a plant-derived dihydroflavonoid. Its main sources are Ampelopsis grossedentata, Hovenia dulcis, Myrica rubra, rhododendrons and some other plants. DHM and related compounds (for example, quercetin) are included due to their anti-inflammatory, antioxidant, hepatoprotective activity. DHM has also shown anticancer and therapeutic activity against asthma, depression and hangovers[Xiang D. et al. Gastrointestinal stability of dihydromyricetin, myricetin, and myricitrin: an in vitro investigation//Int. J. Food Sci. Nutr. 2017. Vol. 68, [symbol] 6. P. 704-711.].

The antioxidant activity of DHM was tested experimentally on HUVEC cells; oxidative stress was modeled using hydrogen peroxide. Pretreatment of cells with DHM (37.5-300 μmol) contributed to a significant decrease in the production of reactive oxygen species, a decrease in the MDA concentration, a decrease in apoptosis by 14.83, 11.1 and 9.12% depending on the dose. The molecular mechanism of DHM action was studied, and its antioxidant activity was found to be associated with the modulation of mitochondrial activity (activation of caspase-9 and caspase-3, release of cytochrome C, inhibition of p53 and PARP)[Hou X. et al. Dihydromyricetin protects endothelial cells from hydrogen peroxide-induced oxidative stress damage by regulating mitochondrial pathways//Life Sci. 2015. Vol. 130. P. 38-46.].

An increased activity of superoxide dismutase, catalase and glutathione peroxidase is another important mechanism of DHM's antioxidant action.[Hou X. et al. Dihydromyricetin protects endothelial cells from hydrogen peroxide-induced oxidative stress damage by regulating mitochondrial pathways//Life Sci. 2015. Vol. 130. P. 38-46.] Experiments on mice with induced diabetes mellitus have shown that DHM improves memory and learning in mice, as well as decreases signs of anxiety and nervousness in comparison with the pathology group without DHM. In addition to reducing the symptoms of oxidative stress, the probable mechanism of DHM action was the induction of the expression of a neurotrophic factor in the brain, which ensures the survival of neurons and the formation of new synapses[Hou X. et al. Dihydromyricetin protects endothelial cells from hydrogen peroxide-induced oxidative stress damage by regulating mitochondrial pathways//Life Sci. 2015. Vol. 130. P. 38-46.].

The anti-hangover effect of DHM is associated with inhibition of the interaction of ethanol with gamma-aminobutyric acid receptors in the nervous system[Liang J., Olsen R. W. Alcohol use disorders and current pharmacological therapies: the role of GABAA receptors//Acta Pharmacol. Sin. 2014. Vol. 35, [symbol] 8. P. 981-993], which contributes to the maintenance of normal cognitive functions, including a decrease in irritability, tendency to depression, etc.

DHM has anti-inflammatory activity proven in a number of animal and human studies. [Chen S. et al. Dihydromyricetin improves glucose and lipid metabolism and exerts anti-inflammatory effects in nonalcoholic fatty liver disease: A randomized controlled trial//Pharmacol. Res. 2015. Vol. 99. P. 74-81.; Chu J. et al. Dihydromyricetin relieves rheumatoid arthritis symptoms and suppresses expression of pro-inflammatory cytokines via the activation of Nrf2 pathway in rheumatoid arthritis model//Int. Immunopharmacol. 2018. Vol. 59. P. 174-180.; Ren Z. et al. Dihydromyricetin exerts a rapid antidepressant-like effect in association with enhancement of BDNF expression and inhibition of neuroinflammation//Psychopharmacology (Berl). 2018. Vol. 235, [symbol] 1. P. 233-244]. DHM mediated reduction of lipid peroxidation, which, in turn, reduced the load on the liver[Ren Z. et al. Dihydromyricetin exerts a rapid antidepressant-like effect in association with enhancement of BDNF expression and inhibition of neuroinflammation//Psychopharmacology (Berl). 2018. Vol. 235, [symbol] 1. P. 233-244.].

The problem was solved due to the qualitative and quantitative composition of the components, which are hydrolysed lignin and dihydromyricetin or quercetin in the stated effective amounts.

### SUMMARY OF THE INVENTION

The invention relates to a composition based on a natural lignocellulose complex, a source of dietary fiber exhibiting antioxidant and adsorption properties, and dihydromyricetin or quercetin used together due to multidirectional action. The claimed composition has an unexpected synergistic effect aimed at reducing the inducers of oxidative stress, manifested in a decreased level of malondialdehyde and acetaldehyde in the blood and human organs, as well as in a beneficial effect on the liver function.

The objective of this invention was to create a new composition for reducing oxidative stress and its inducers in blood and human organs, as well as maintaining liver functions, which contains a composition of a lignocellulose complex and dihydromyricetin or quercetin and has high stability, without limitations on use in all age groups and specific groups patients, due to the synergistic effect of its active components presented as an oral dosage form, as a biologically active food supplement, as a product of therapeutic or prophylactic dietary nutrition, and also as a food product (food).

One of the embodiments of the invention disclosed herein is an oral dosage form of tablets, powder, gel or suspension and other dosage forms containing a lignocellulose complex and and dihydromyricetin or quercetin.

Another embodiment of this invention disclosed herein is a dietary supplement used to reduce oxidative stress and its inducers in the blood and human organs, as well as to maintain liver function, which contains a lignocellulose complex and dihydromyricetin or quercetin.

Another embodiment of the invention is a product for therapeutic and prophylactic dietary nutrition used to reduce oxidative stress and its inducers in the blood and human organs, as well as to maintain liver functions, which contains a lignocellulose complex and dihydromyricetin or quercetin.

Another embodiment of the claimed invention is the above combination with the additional containt of glycine and thiamine. Glycine can represent derivatives and/or pharmaceutically acceptable salts of glycine in an amount of 50 mg to 1000 mg, while thiamine is a derivative of thiamine, for example benfotiamine and/or pharmaceutically acceptable salts in an amount of 1 mg to 500 mg.

The components present in the composition have different mechanisms of antioxidant, anti-inflammatory, hepatoprotective action, which together unexpectedly led to the ability to effectively correct the observed disorders, including increased MDA and AA levels, liver enzyme levels, pro-inflammatory factors, a decreased activity of the antioxidant system, damage to the liver tissue structure that provoke reduced functions (foci of necrosis, fibrosis).

In a preferred embodiment, the invention relates to a composition in the form of an oral dosage form for reducing oxidative stress and its inducers in the blood and human organs, as well as maintaining liver functions, which contains hydrolytic lignin in the amount from 50 to 5000 mg and dihydromyricetin or quercetin in the amount of 5 to 1000 mg, which corresponds to the effective daily dosage. In the most preferred embodiment of the invention, the weight of the said composition provides for an effective dosage of hydrolytic lignin from 4000 to 5000 mg per day and dihydromyricetin or quercetin from 900 to 1000 mg per day.

Further, the invention relates to using the said composition as an oral dosage form in effective amounts for the complex maintenance of the detoxification function and restoration of the liver.

The invention can be embodied as a biologically active food supplement that contains the specified composition and various acceptable additives. In this case, the composition weight provides for a dosage of 50 to 5000 mg per day of hydrolytic lignin and from 5 to 1000 mg of dihydromyricetin or quercetin.

Further, the invention relates to using a dietary supplement for the complex maintenance of the detoxification function and restoration of the liver.

In yet another embodiment of the invention, a product is provided for effective therapeutic and prophylactic dietary nutrition, which contains the specified composition and acceptable additives.

The invention also relates to using a product for therapeutic and prophylactic dietary nutrition, which contains the specified composition in effective amounts for the liver treatment and acceptable additives for the complex maintenance of the detoxification function and restoration of the liver. In this case, the composition weight provides for a dosage of 50 to 5000 mg per day of hydrolytic lignin and from 5 to 1000 mg of dihydromyricetin or quercetin. In the most preferred embodiment of the invention, the weight of the said composition provides for an effective dosage of hydrolytic lignin from 4000 to 5000 mg per day and dihydromyricetin or quercetin from 900 to 1000 mg per day.

The above composition was unexpectedly found to contribute to preserving the functional activity of the liver due to a protective effect against pathological, including necrotic changes in the liver structure by implementing a complex multidirectional protective effect, namely, by reducing oxidative stress and lipid peroxidation, reducing the effect of inflammatory cytokines and toxic agents.

Using the above combination has a modulating effect, including on alcohol metabolism, which manifests itself in the reduced negative effects of ethanol and its metabolic products on the body, including liver and nervous system, as well as on the general clinical state, physical activity and cognitive functions. The protective effect is primarily associated with the suppressed negative effect of oxidative stress and a decreased level of oxidative stress inducers in the body, as well as a decreased negative effect of acetaldehyde and other ethanol decomposition products on the liver function.

At the same time, animal studies have shown that adding dihydromyricetin or quercetin to the composition increases the antioxidant capacity of the blood of experimental animals and reduces the general inflammation in the liver tissues. DHM increases the activity of the antioxidant system enzymes, such as superoxide dismutase, catalase and glutathione peroxidase, reducing the MDA level and the production of reactive oxygen species, as well as inhibiting the production of pro-inflammatory factors (TNFα, pro-inflammatory cytokines). DHM promotes the activation of the ethanol metabolism system enzymes, thereby ensuring a faster alcohol and acetaldehyde transformation and reducing the adverse effect of these substances on the body cells and organs.

The inclusion of the lignocellulose complex makes it possible to adsorb fat and bile acids in the intestine, preventing their absorption into the blood. In addition, due to its antioxidant effect, the lignocellulose complex apparently reduces lipid peroxidation in intestinal cells, which leads to a decreased permeability of the intestinal wall for bacterial lipopolysaccharides and toxins that induce liver inflammatory reactions. The direct adsorption effect of lignin helps reduce the toxic load on the liver.

Thus, the technical result of the claimed composition, intended to reduce oxidative stress and the level of its inducers in the blood, also aimed at maintaining liver functions, is to achieve an unexpected synergistic effect obtained through the implementation of the mechanism of the simultaneous effect of dihydromyricetin or quercetin on the functions of the liver and intestines in the declared effective amounts in conjunction with the pronounced adsorption and antioxidant activity of hydrolysis lignin in the form of a ligninocellulose complex in the declared effective amounts.

A decreased oxidative stress and its inducers is manifested, respectively, in a decreased level of malondialdehyde and acetaldehyde in the blood and organs, as well as in a beneficial effect on liver functions.

When used in effective amounts, the combination of the mentioned components can be used to reduce hepatotoxicity as a result of drug intoxication or alcohol intoxication.

### EXEMPLARY EMBODIMENTS

### Example 1

This example illustrates the preparation of a composition containing lignin and DHM or quercetin.

To obtain the composition, the following procedures were carried out:
1) lignin grinding using a mill with 0.8 mm mesh size.
2) sieving of substances—to remove foreign inclusions and lumps, substances were sifted using a vibrating sieve with a 0.32 mm mesh size. Each component was sieved separately. The sifted raw material was re-weighed. The amount of dropout should not exceed 6 wt %.
3) preparation of compositions—the amount of active ingredients was calculated, including: hydrolysed lignin in an amount that allows for a dosage of lignin from 50 to 5000 mg/day, DHM in an amount that allows for a dosage of DHM from 5 to 1000 mg/day, quercetin in an amount that allows you to provide a dosage of quercetin from 5 to 1000 mg/day. Raw materials were weighed. The raw material was loaded in portions into the mixer, the resulting mass was mixed for 20 minutes at a mixer rotation speed of 15-20 rpm until the components were evenly distributed. The resulting composition was packaged in a sealed container made of opaque glass or plastic, marking was applied indicating the ratio of active components, the date of production and storage conditions. For further studies, samples weighing 500 g were obtained: 1) composition 1-416.667 g of lignin and 83.333 g of DHM, which is equivalent to a dosage of lignin, 5000 mg+DHM, 1000 mg; 2) composition 2-446.439 g of lignin and 53.571 g of DHM, which is equivalent to a dosage of lignin, 2500 mg+DHM, 300 mg; 3) composition 3-471.698 g of lignin and 28.302 g of DHM, which is equivalent to the dosage of lignin, 500 mg+DHM, 30 mg; 4) composition 4-471.675 g of lignin and 28.325 g of DHM, which is equivalent to the dosage of lignin, 250 mg+DHM, 15 mg; 5) composition 5-454.545 g of lignin and 45.455 g of DHM, which is equivalent to the dosage of lignin, 50 mg+DHM, 5 mg; 6) composition 6-416.667 g of lignin and 83.333 g of quercetin, which is equivalent to the dosage of lignin, 5000 mg+quercetin, 1000 mg; 7) composition 7-446.439 g of lignin and 53.571 g of quercetin, which is equivalent to the dosage of lignin, 2500 mg+quercetin, 300 mg; 8) composition 8-471.698 g of lignin and 28.302 g of quercetin, which is equivalent to the dosage of lignin, 500 mg+quercetin, 30 mg; 9) composition 9-471.675 g of lignin and 28.325 g of quercetin, which is equivalent to the dosage of lignin, 250 mg+quercetin, 15 mg; 10) composition 10-454.545 g of lignin and 45.455 g of quercetin, which is equivalent to the dosage of lignin, 50 mg+quercetin, 5 mg.

### Example 2

The example illustrates preparing the combinations of lignin and DHM/quercetin with the addition of auxiliary glycine and thiamine components.

To obtain compositions of lignin, DHM, glycine and thiamine, as well as compositions of lignin, quercetin, glycine and thiamine, the following procedures were performed:
1) grinding of lignin and glycine using a mill with a 0.8 mm mesh size.
2) sieving of substances—to remove foreign inclusions and lumps, substances were sifted using a vibrating sieve with a 0.32 mm mesh size. Each component was sieved separately. The sifted raw material was re-weighed. The amount of dropout should not exceed 6 wt %.
3) obtaining compositions—the amount of active ingredients was calculated, including: hydrolysed lignin in an amount that allows for a dosage of lignin from 50 to 5000 mg/day, DHM in an amount that allows a dosage of DHM from 5 to 1000 mg/day, quercetin in an amount that allows to provide a dosage of quercetin from 5 to 1000 mg/day, glycine in an amount that allows a dosage of glycine from 50 to 1000 mg/day, thiamine in an amount that allows a dosage of glycine from 1 to 500 mg/day. Raw materials were weighed. The raw material was loaded in portions into the mixer, the resulting mass was mixed for 20 minutes at a mixer rotation speed of 15-20 rpm until the components were evenly distributed. The resulting composition was packaged in a sealed container made of opaque glass or plastic, marking was applied indicating the ratio of active components, the date of production and storage conditions. For further studies, samples weighing 500 g were obtained:
1) composition 11-384.615 g of lignin, 76.923 g of DHM and 38.462 g of thiamine, which is equivalent to the dosage of lignin, 5000 mg+DHM, 1000 mg+thiamine, 500 mg;
2) composition 12-384.615 g of lignin, 76.923 g of quercetin and 38.462 g of thiamine, which is equivalent to the dosage of lignin, 5000 mg+quercetin, 1000 mg+thiamine, 500 mg;
3) composition 13-438.596 g of lignin, 52.632 g of DHM and 8.772 g of thiamine, which is equivalent to the dosage of lignin, 2500 mg+DHM, 300 mg+thiamine, 50 mg;
4) composition 14-438.596 g of lignin, 52.632 g of quercetin and 8.772 g of thiamine, which is equivalent to the dosage of lignin, 2500 mg+quercetin, 300 mg+thiamine, 50 mg;
5) composition 15-467.254 g of lignin, 28.035 g of DHM and 4.673 g of thiamine, which is equivalent to the dosage of lignin, 500 mg+DHM, 30 mg+thiamine, 5 mg;
6) composition 16-467.254 g of lignin, 28.035 g of quercetin and 4.673 g of thiamine, which is equivalent to the dosage of lignin, 500 mg+quercetin, 30 mg+thiamine, 5 mg;
7) composition 17-468.150 g of lignin, 28.089 g of DHM and 3.745 g of thiamine, which is equivalent to the dosage of lignin, 250 mg+DHM, 15 mg+thiamine, 2 mg;
8) composition 18-468.150 g of lignin, 28.089 g of quercetin and 3.745 g of thiamine, which is equivalent to the dosage of lignin, 250 mg+quercetin, 15 mg+thiamine, 2 mg;
9) composition 19-446.425 g of lignin, 44.643 g of DHM and 8.929 g of thiamine, which is equivalent to the dosage of lignin, 50 mg+DHM, 5 mg+thiamine, 1 mg;
10) composition 20-446.425 g of lignin, 44.643 g of quercetin and 8.929 g of thiamine, which is equivalent to the dosage of lignin, 50 mg+quercetin, 15 mg+thiamine, 1 mg;
11) composition 21-357.143 g of lignin, 71.429 g of DHM and 71.429 g of glycine, which is equivalent to the dosage of lignin, 5000 mg+DHM, 1000 mg+glycine, 1000 mg;
12) composition 22-357.143 g of lignin, 71.429 g of quercetin and 71.429 g of glycine, which is equivalent to the dosage of lignin, 5000 mg+quercetin, 1000 mg+glycine, 1000 mg;
13) composition 23-378.788 g of lignin, 45.455 g of DHM and 75.757 g of glycine, which is equivalent to the dosage of lignin, 2500 mg+DHM, 300 mg+glycine, 500 mg;
14) composition 24-378.788 g of lignin, 45.455 g of quercetin and 75.757 g of glycine, which is equivalent to the dosage of lignin, 2500 mg+quercetin, 300 mg+glycine, 500 mg;
15) composition 25-320.500 g of lignin, 19.231 g of DHM and 160.259 g of glycine, which is equivalent to the dosage of lignin, 500 mg+DHM, 30 mg+glycine, 250 mg;
16) composition 26-320.500 g of lignin, 19.231 g of quercetin and 160.250 g of glycine, which is equivalent to the dosage of lignin, 500 mg+quercetin, 30 mg+glycine, 259 mg;
17) composition 27-333.333 g of lignin, 66.667 g of DHM, 33.333 g of thiamine and 66.667 g of glycine, which is equivalent to the dosage of lignin, 5000 mg+DHM, 1000 mg+thiamine, 500 mg+glycine, 1000 mg;
18) composition 28-333.333 g of lignin, 66.667 g of quercetin, 33.333 g of thiamine and 66.667 g of glycine, which is equivalent to the dosage of lignin, 5000 mg+quercetin, 1000 mg+thiamine, 500 mg+glycine, 1000 mg;
19) composition 29-373.134 g of lignin, 44.776 g of DHM, 7.463 g of thiamine and 74.627 g of glycine, which is equivalent to the dosage of lignin, 2500 mg+DHM, 300 mg+thiamine, mg+glycine, 500 mg;
20) composition 30-373.134 g of lignin, 44.776 g of quercetin, 7.463 g of thiamine and 74.627 g of glycine, which is equivalent to the dosage of lignin, 2500 mg+quercetin, 300 mg+thiamine, 50 mg+glycine, 500 mg;
21) composition 31-318.450 g of lignin, 19.107 g of DHM, 3.185 g of thiamine and 159.225 g of glycine, which is equivalent to the dosage of lignin, 500 mg+DHM, 30 mg+thiamine, 5 mg+glycine, 250 mg;
22) composition 32-318.450 g of lignin, 19.107 g of quercetin, 3.185 g of thiamine and 159.225 g of glycine, which is equivalent to the dosage of lignin, 500 mg+quercetin, 30 mg+thiamine, 5 mg+glycine, 250 mg;
23) composition 33-340.575 g of lignin, 20.435 g of DHM, 2.725 g of thiamine and 136.230 g of glycine, which is equivalent to the dosage of lignin, 250 mg+DHM, 15 mg+thiamine, 2 mg+glycine, 100 mg;
24) composition 34-340.575 g of lignin, 20.435 g of quercetin, 2.725 g of thiamine and 136.230 g of glycine, which is equivalent to the dosage of lignin, 250 mg+quercetin, 15 mg+thiamine, 2 mg+glycine, 100 mg;
25) composition 35-235.845 g of lignin, 23.585 g of DHM, 4.717 g of thiamine and 235.845 g of glycine, which is equivalent to the dosage of lignin, 50 mg+DHM, 5 mg+thiamine, 1 mg+glycine, 50 mg;
26) composition 36-235.845 g of lignin, 23.585 g of quercetin, 4.717 g of thiamine and 235.845 g of glycine, which is equivalent to the dosage of lignin, 50 mg+quercetin, 5 mg+thiamine, 1 mg+glycine, 50 mg.

### Example 3

This example illustrates the study of the claimed composition in relation to drug hepatotoxicity.

Many commonly used drugs, including paracetomol, the well-known analgesic and antipyretic drug, exhibit liver toxicity. An overdose of paracetomol causes acute hepatotoxicity, including the formation of necrosis foci.

Procedure: Outbred male rats weighing 250-270 g were studied. The study involved 11 groups of animals, 6 animals in each group: group 1—pathology control, group 2—lignin, 5000 mg+DHM, 1000 mg, group 3—lignin, 2500 mg+DHM, 300 mg, group 4—lignin, 500 mg+DHM, 30 mg, group 5 lignin, 5000 mg+DHM, 1000 mg+glycine, 1000 mg, group 6-lignin, 5000 mg+DHM, 1000 mg+thiamine, 500 mg, group 7—lignin, 5000 mg+DHM, 1000 mg+thiamine, 500 mg+glycine, 1000 mg, group 8—lignin, 250 mg+DHM, 15 mg+thiamine, 2 mg+glycine, 100 mg, group 9—lignin, 50 mg+DHM, 5 mg, group 10—lignin, mg+DHM, 5 mg+thiamine, 1 mg+glycine, 50 mg, group 11—lignin, 5000 mg+quercetin, 1000 mg. The dosages are indicated according to the human normal values; when calculating the doses, a recalculation was made taking into account the weight and metabolic coefficient for experimental animals. Before the start of the experiment, the animals were deprived of access to food for 12 hours. Paracetamol as a suspension in 1% starch solution was administered intragastrically using a probe. The paracetomol dosage was 2 g/kg body weight. One hour after the introduction of paracetamol, the studied combinations were also introduced in the form of suspensions in a starch solution. Two hours after the introduction of the studied substances, the animals received a standard food. The observation was continued for 24 hours, after which the animals were euthanized. During the study, ALT, AST, bilirubin, MDA and TNFα blood levels were determined in animals, and the liver was subjected to histological examination at the end of the study. The degree of liver damage was assessed by summing the number and size of necrotic foci and inflammation foci during microscopic analysis using a scoring system.

Results: Paracetomol overdose led to significant functional changes in the liver: increased ALT, AST and bilirubin levels, significant oxidative stress, which led to an increased blood MDA, increased proinflammatory cytokine TNFα level, as well as the appearance of necrosis foci in the liver tissues (Table. 1, mean value±standard deviation). All combinations studied contributed to preserving liver functions, which was expressed in a decreased severity of pathological changes in liver tissues (calculated in points), as well as a decreased level of liver enzymes (ALT and AST) and bilirubin. Against the background of using the studied combinations, a decreased intensity of oxidative stress caused by paracetomol overdose was observed, which was manifested in a decreased malondialdehyde level. A significant decrease in the pro-inflammatory cytokine TNFα level, which was observed against the background of using the mentioned combinations, allows concluding on their anti-inflammatory activity, which is also of great importance for maintaining the normal liver functioning and the body's resistance against oxidative stress. The most pronounced beneficial effect was shown by a combination of lignin, 5000 mg+DHM, 1000 mg and a combination of lignin, 5000 mg+quercetin, 1000 mg. Glycine and/or thiamine introduced into the LH and DHM combination, and, in particular, their mixtures, enhanced the protective effect of the combination. The data obtained indicate that using a combination of lignin at a dose of 5000 mg and DHM (quercetin) at a dose of 1000 mg allowed achieving the most pronounced anti-toxic effect of paracetomol in relation to the liver, which makes it possible to preserve liver function.

TABLE 1
Data of biochemical and histological analysis before the start of the study (using
the example of the control group) and after loading with paracetamol (2 g/kg)

| | ALT, | AST, | Bilirubin, | MDA, | Liver damage, | TNFα level, |
|---|---|---|---|---|---|---|
| Groups: | u/l | u/l | mg/dl | nmol/mg | points | pg/ml |
| Control (prior to the beginning | 53.18 ± 6.36* | 87.82 ± 7.81* | 0.194 ± 0.009* | 1.46 ± 0.23* | 0*** | 34.60 ± 2.60* |
| of the study) | | | | | | |
| Control (at the end of the study) | 193.98 ± 12.25^{#} | 265.43 ± 12.73^{#} | 0.246 ± 0.005^{#} | 3.33 ± 0.23^{#} | 17.33 ± 3.39^{#} | 170.42 ± 10.33^{#} |
| lignin, 5000 mg + DHM, | 66.47 ± 4.12*^{#} | 105.28 ± 5.09*^{#} | 0.170 ± 0.006*^{#} | 1.79 ± 0.17*^{#} | 8.67 ± 2.07*^{#} | 65.29 ± 17.23* |
| 1000 mg | | | | | | |
| lignin, 2500 mg + DHM, | 92.12 ± 5.27*^{#} | 152.92 ± 6.13*^{#} | 0.228 ± 0.003*^{#} | 2.35 ± 0.08*^{#} | 11.33 ± 3.01*^{#} | 95.87 ± 7.03* |
| 300 mg | | | | | | |
| lignin, 500 mg + DHM, 30 mg | 112.65 ± 6.03*^{#} | 186.70 ± 8.08*^{#} | 0.215 ± 0.004*^{#} | 2.78 ± 0.16*^{#} | 14.17 ± 4.58^{#} | 113.33 ± 6.07* |
| lignin, 5000 mg + DHM, 1000 | 61.65 ± 3.33* | 101.05 ± 9.75* | 0.164 ± 0.004*^{#} | 1.63 ± 0.12* | 8.83 ± 1.94*^{#} | 62.61 ± 4.82* |
| mg + glycine, 1000 mg | | | | | | |
| lignin, 5000 mg + DHM, 1000 | 61.85 ± 5.51* | 99.93 ± 6.02* | 0.161 ± 0.006*^{#} | 1.62 ± 0.16* | 8.33 ± 1.37*^{#} | 60.53 ± 6.92* |
| mg + thiamine, 500 mg | | | | | | |
| lignin, 5000 mg + DHM, 1000 | 59.98 ± 4.37* | 94.27 ± 5.21* | 0.154 ± 0.004*^{#} | 1.57 ± 0.14* | 7.68 ± 12.01*^{#} | 57.16 ± 14.36* |
| mg + thiamine, 500 mg + | | | | | | |
| glycine, 1000 mg | | | | | | |
| lignin, 250 mg + DHM, 15 mg + | 146.17 ± 12.25*^{#} | 209.47 ± 9.95*^{#} | 0.231 ± 0.005*^{#} | 2.94 ± 0.18*^{#} | 14.92 ± 3.71^{#} | 124.56 ± 6.20* |
| thiamine, 2 mg + glycine, | | | | | | |
| 100 mg | | | | | | |
| lignin, 50 mg + DHM, 5 mg | 186.11 ± 8.13^{#} | 247.02 ± 10.24^{#} | 0.234 ± 0.006^{#} | 3.20 ± 0.13^{#} | 16.72 ± 2.30^{#} | 159.34 ± 6.23* |
| lignin, 50 mg + DHM, 5 mg + | 183.54 ± 10.18^{#} | 241.93 ± 14.87^{#} | 0.239 ± 0.008^{#} | 3.15 ± 0.21^{#} | 16.09 ± 2.98^{#} | 157.12 ± 5.36* |
| thiamine, 1 mg + glycine, 50 mg | | | | | | |
| lignin, 5000 mg + quercetin, | 67.32 ± 3.25*^{#} | 107.27 ± 5.11*^{#} | 0.178 ± 0.007*^{#} | 1.92 ± 0.16*^{#} | 9.17 ± 1.94*^{#} | 65.52 ± 7.80* |
| 1000 mg | | | | | | |

*p < 0.05 versus the control group at the end of the study
^{#}p < 0.05 versus the control group before the start of the study

### Example 4

This example illustrates the study of the claimed composition in relation to alcohol intoxication of the human body.

The description presents various diseases and conditions characterized by elevated blood MDA and AA levels. The most well-developed models for restoring such conditions are the models with a single or multiple administration of alcohol to laboratory animals, mainly rats. Given the reliability of alcoholic models, a model of this kind was chosen to confirm the studied combinations' ability to reduce blood MDA and AA levels when they increase due to various reasons. Wistar rats weighing 200-220 g were studied. The study involved 11 groups of animals, 10 rats in each group: group 1—pathology control, group 2—lignin, 5000 mg+DHM, 1000 mg, group 3—lignin, 2500 mg+DHM, 300 mg, group 4—lignin, 500 mg+DHM, 30 mg, group 5—lignin, 5000 mg+DHM, 1000 mg+glycine, 1000 mg, group 6—lignin, 5000 mg+DHM, 1000 mg+thiamine, 500 mg, group 7—group 7—lignin, 5000 mg+DHM, 1000 mg+thiamine, 500 mg+glycine, 1000 mg, group 8—lignin, 250 mg+DHM, 15 mg+thiamine, 2 mg+glycine, 100 mg, group 9—lignin, 50 mg+DHM, 5 mg, group 10—lignin, 50 mg+DHM, 5 mg+thiamine, 1 mg+glycine, 50 mg, group 11—lignin, 5000 mg+quercetin, 1000 mg. The dosages are indicated according to the human normal values; when calculating the doses, a recalculation was made taking into account the weight and metabolic coefficient for experimental animals. The investigated substances and their mixtures were injected intragastrically using a probe in the form of suspensions prepared in 1% starch solution, 30 minutes before the introduction of ethanol. Ethanol solution was introduced to rats intragastrically using a probe, based on a dosage of 12 ml of 40% ethanol solution per 1 kg of body weight. The ethanol solution was administered fractionally in 4 doses with an interval of 1 hour between the injections. The individual volume of the injected solution was calculated in accordance with the body weight of the animal, measured immediately before administration. Blood sampling for studying the analyzed parameters (the level of ethanol, AA and MDA, as well as the level of the antioxidant enzyme superoxide dismutase (SOD) in the blood) was performed before the start of the study, then 1 hour after the last injection of ethanol and 8 hours after the last injection of ethanol. The experimental results are presented in Table 2.

TABLE 2
Content of ethanol, malondialdehyde (MDA), acetaldehyde (AA) and superoxide dismutase (SOD) in the blood
of rats that received the selected substances 30 minutes before the introduction of 40% ethanol solution

| | Ethanol level, mg/ml | | | MDA level, nM | | | AA level, μM | | | SOD level, u/mg | |
|---|---|---|---|---|---|---|---|---|---|---|---|
| Groups: | 0 h | 1 h | 8 h | 0 h | 1 h | 8 h | 0 h | 1 h | 8 h | 0 h | 8 h |
| Control | 0 | 6.79 ± | 0.03 ± | 7.36 ± | 12.08 ± | 9.33 ± | 71.37 ± | 120.52 ± | 79.49 ± | 25.55 ± | 8.52 ± |
| | | 0.25^{#} | 0.00 | 0.41 | 0.37^{#} | 0.26 | 2.21 | 5.05 | 2.26 | 2.70 | 0.84^{#} |
| lignin, 5000 mg + | 0 | 4.68 ± | 0.02 ± | 7.22 ± | 3.74 ± | 5.12 ± | 72.53 ± | 83.93 ± | 69.67 ± | 24.91 ± | 22.62 ± |
| DHM, 1000 mg | | 0.25^{#}* | 0.00 | 0.38 | 0.09^{#}* | 0.13^{#}* | 2.21 | 3.30* | 2.73 | 3.01 | 1.59* |
| lignin, 2500 mg + | 0 | 5.37 ± | 0.03 ± | 6.95 ± | 4.31 ± | 6.02 ± | 71.23 ± | 102.36 ± | 68.30 ± | 25.01 ± | 17.18 ± |
| DHM, 300 mg | | 0.19^{#}* | 0.00 | 0.24 | 0.11^{#}* | 0.25* | 2.58 | 2.63*^{#} | 2.91 | 3.10 | 0.94* |
| lignin, 500 mg + | 0 | 5.83 ± | 0.03 ± | 7.03 ± | 8.96 ± | 6.23 ± | 78.15 ± | 116.89 ± | 80.5 ± | 25.26 ± | 12.49 ± |
| DHM, 30 mg | | 0.34^{#}* | 0.00 | 0.15 | 0.68^{#}* | 0.55 | 2.43 | 5.52^{#} | 3.37 | 3.18 | 0.87* |
| lignin, 5000 mg + | 0 | 4.44 ± | 0.02 ± | 7.40 ± | 3.95 ± | 4.75 ± | 73.23 ± | 80.79 ± | 69.24 ± | 25.68 ± | 23.17 ± |
| DHM, 1000 mg + | | 0.24^{#} | 0.00 | 0.22 | 0.28^{#}* | 0.28^{#}* | 1.35 | 1.71* | 1.46 | 3.32 | 1.19* |
| glycine, 1000 mg | | | | | | | | | | | |
| lignin, 5000 mg + | 0 | 4.47 ± | 0.02 ± | 7.07 ± | 3.99 ± | 4.60 ± | 72.30 ± | 79.07 ± | 69.63 ± | 25.02 ± | 24.00 ± |
| DHM, 1000 mg + | | 0.20^{#} | 0.00 | 0.34 | 0.30^{#}* | 0.27^{#}* | 2.43 | 1.58* | 2.23 | 3.11 | 1.16* |
| thiamine, 500 mg | | | | | | | | | | | |
| lignin, 5000 mg + | 0 | 4.17 ± | 0.02 ± | 7.28 ± | 3.65 ± | 4.60 ± | 74.62 ± | 75.89 ± | 70.51 ± | 25.32 ± | 25.62 ± |
| DHM, 1000 mg + | | 0.19^{#} | 0.00 | 0.27 | 0.23^{#}* | 0.27^{#}* | 2.07 | 2.36* | 1.94 | 3.17 | 2.04* |
| thiamine, 500 mg + | | | | | | | | | | | |
| glycine, 1000 mg | | | | | | | | | | | |
| lignin, 250 mg + | 0 | 6.02 ± | 0.02 ± | 6.96 ± | 9.34 ± | 6.86 ± | 71.89 ± | 118.07 ± | 78.45 ± | 25.07 ± | 15.87 ± |
| DHM, 15 mg + | | 0.21^{#}* | 0.00 | 0.22 | 0.42^{#}* | 0.39* | 2.17 | 4.36^{#} | 3.08 | 3.44 | 1.14* |
| thiamine, 2 mg + | | | | | | | | | | | |
| glycine, 100 mg | | | | | | | | | | | |
| lignin, 50 mg + | 0 | 6.53 ± | 0.03 ± | 7.20 ± | 11.02 ± | 8.43 ± | 74.67 ± | 119.63 ± | 79.91 ± | 24.95 ± | 9.93 ± |
| DHM, 5 mg | | 0.12^{#} | 0.00 | 0.25 | 0.38^{#}* | 0.26^{#}* | 2.71 | 4.56^{#} | 2.09 | 3.15 | 0.78^{#} |
| lignin, 50 mg + | 0 | 6.49 ± | 0.03 ± | 7.21 ± | 10.68 ± | 8.21 ± | 75.11 ± | 119.45 ± | 79.92 ± | 24.89 ± | 10.54 ± |
| DHM, 5 mg + | | 0.17^{#} | 0.00 | 0.30 | 0.54^{#}* | 0.30^{#}* | 3.01 | 4.97^{#} | 2.19 | 3.26 | 0.92^{#} |
| thiamine, 1 mg + | | | | | | | | | | | |
| glycine, 50 mg | | | | | | | | | | | |
| lignin, 5000 mg + | 0 | 4.80 ± | 0.02 ± | 7.14 ± | 4.45 ± | 5.44 ± | 74.72 ± | 87.66 ± | 71.95 ± | 24.97 ± | 21.92 ± |
| quercetin, 1000 mg | | 0.15^{#}* | 0.00 | 0.24 | 0.15^{#}* | 0.16^{#}* | 1.73 | 1.64* | 2.31 | 3.09 | 1.30* |

*p < 0.05 in relation to the Control group at the same time point (nonparametric Mann-Whitney test),
^{#}p□0.05 relative to the value before ethanol administration in the same group (Wilcoxon nonparametric test).

As shown in Table 2, all the combinations studied have an effect on the ethanol blood level 1 hour after the last administration, and also contribute to a decrease in oxidative stress and lipid peroxidation after the administration of ethanol (1 and 8 hours after the last administration). The studied combinations contributed to a decrease in the MDA level in all groups. The SOD level significantly decreased against the background of introducing ethanol in the control group. However, the enzyme level in the groups receiving the studied combinations was significantly higher than in the control group, which indicates a more active antioxidant defense in the animals' organism. The use of combinations made it possible to achieve a significant decrease in the acetaldehyde level—one of the key inducers of oxidative stress and liver damage while taking ethanol. The most pronounced effect was observed with the introduction of a combination of lignin, 5000 mg+DHM, 1000 mg, when using thiamine and/or glycine as additional components, as well as with the introduction of a combination of lignin, 5000 mg+quercetin, 1000 mg. The results obtained indicate a comparable efficacy of the combinations of lignin, 5000 mg+DHM, 1000 mg and lignin, 5000 mg+quercetin, 1000 mg.

The beneficial effect of thiamine is associated with its ability to activate various branches of the body's antioxidant system, as well as activate many biochemical processes, thereby contributing to the accelerated elimination of ethanol and its decay products from the body. The beneficial effect of glycine is associated with its ability to interact with gamma-aminobutyric acid receptors in the nervous system, which reduces the central nervous system's sensitivity to the effects of ethanol. In addition, glycine has antihypoxic and antioxidant effects, reduces the hepatocytes and Kupffer cells' sensitivity to the effects of endotoxins, and also reduces their ability to synthesize pro-inflammatory cytokines.

### Example 5

Obtaining finished forms of food additives and food products.

It should be noted that the above studies on a composition containing lignin, dihydromyricetin or quercetin were carried out using ready-made forms containing auxiliary components, which in turn were used to reduce oxidative stress and the level of oxidative stress inducers in the blood and organs, as well as for the complex maintenance of detoxification function and restoration of the liver.

Based on the disclosed composition of lignin and dihydromyricetin or quercetin, the invention can be embodied as a biologically active food additive or product for therapeutic and prophylactic dietary nutrition in the form of tablets, for the creation of which auxiliary substances were used from the following range: stabilizers, fillers, anti-caking and binding agents: hemicellulose, guar gum, acacia gum, gum arabic, locust bean gum, starches, including starch esters and modified starches, dextrins, pectins, lecithins, polydextroses, cellulose, including modified celluloses, croscarmellose dihydroxide, polyviniroses, polyviniroses magnesium aluminum silicates, calcium carbonate, lactose monohydrate; leavening agents: povidone and its modifications, starches, including modified, cellulose, including modified; acidity regulators: acetic acid and its salts, ascorbic acid and its salts, citric acid and its salts, lactic acid and its salts, malic acid and its salts, tartaric acid and its salts; glidants: talc, starch, including modified starch, silicon dioxide, macrogol, stearic acid and its salts; dyes: curcumin, anthocyanins, carmine, betanin, capsorubin; sweeteners: aspartame, acesulfame potassium, polyols, stevia components, sucralose, glycyrrhizic acid and its salts; antioxidants: ascorbic acid and its salts, citric acid and its salts, tocopherols, tartaric acid and its salts; preservatives: sorbic acid and its salts, benzoic acid and its salts, acetic acid and its salts, propionic acid and its salts; glazers: wax (beeswax, candle wax, microcrystalline, polyethylene) and shellac. The selected auxiliary substances were used in quantities that ensure their technological properties and characteristics required for the finished form.

Based on the disclosed composition of lignin and dihydromyricetin or quercetin, the invention can be embodied as a biologically active food additive or food product in the form of a powder, for the creation of which the following auxiliary substances were used: stabilizers, fillers, anticaking agents: hemicellulose, starches, including starch esters and modified starches, dextrins, pectins, lecithins, polydextroses, cellulose, including modified cellulose, croscarmellose, polyvinylpyrrolidone, silicon dioxide, magnesium silicates, calcium carbonate, lactose monohydrate; acidity regulators: acetic acid and its salts, ascorbic acid and its salts, citric acid and its salts, lactic acid and its salts, malic acid and its salts, tartaric acid and its salts, hydrochloric acid, hydrogen peroxide; colorants: curcumin, anthocyanins, carmine, betanin, tannins, capsorubin; sweeteners: aspartame, acesulfame potassium, polyols, stevia components, sucralose, glycyrrhizic acid and its salts; glidants: talc, starch, including modified starch, silicon dioxide, macrogol, stearic acid and its salts; antioxidants: ascorbic acid and its salts, citric acid and its salts, tocopherols, tartaric acid and its salts; preservatives: sorbic acid and its salts, benzoic acid and its salts, acetic acid and its salts, propionic acid and its salts; commercially available flavors. The selected auxiliary substances were used in quantities that ensure their technological properties and characteristics required for the finished form.

Based on the disclosed composition of lignin and dihydromyricetin or quercetin, the invention can be embodied as a biologically active food supplement or food product in the form of a bar, candy or snack, which was created using auxiliary substances from the following range: fillers (base)—puree from fresh or dried fruits and vegetables, fresh or dried fruit and vegetable paste, muesli, corn, rice and cereal flakes, corn, rice and cereal extruded balls, soy protein isolate, milk protein concentrate, sugar syrups, including glucose-fructose syrup, sugar-milk syrup, fruit and berry syrup, animal and vegetable fat; flavors: flavors permitted for use in food, fresh and dried berries, fruits and vegetables, coconut, nuts and seeds, honey, cocoa powder, chocolate, salt, spices, citric acid and its salts; antioxidants: ascorbic acid and its salts, citric acid and its salts, tocopherols, tartaric acid and its salts. The selected auxiliary substances were used in quantities that ensure their technological properties and characteristics required for the finished form.

1. The combination for reducing oxidative stress and its inducers in the blood and human organs, which contains dihydromyricetin or quercetin in an amount of 5 to 1000 mg and hydrolytic lignin in an amount of 50 mg to 5000 mg.

2. The combination according to claim 1, wherein the preferred content of dihydromyricetin or quercetin is in an amount from 50 to 1000 mg and hydrolytic lignin in an amount from 500 mg to 5000 mg.

3. The combination according to claim 1, wherein it additionally contains glycine, thiamine.

4. The combination according to claim 3, wherein the glycine is a derivative and/or pharmaceutically acceptable salt of glycine in an amount from 50 mg to 1000 mg.

5. The combination according to claim 3, wherein thiamine is a thiamine derivative, for example benfotiamine, and/or pharmaceutically acceptable salts in an amount of 1 mg to 500 mg.

6. The combination according to claim 1, wherein it is an orally dosage form, namely powder, tablets, capsules, suspensions.

7. The use of the combination according to claim 1 for the comprehensive maintenance of the detoxification function and restoration of the liver.

8. Biologically active food supplement containing the composition according to claim 1 and auxiliary substances.

9. Biologically active food supplement according to claim 8, wherein it is made in the form of a powder, tablet, capsule, bar, and snack.

10. The use of biologically active food supplement according to claim 8 for the comprehensive maintenance of the detoxification function and restoration of the liver.

11. A product for therapeutic and prophylactic dietary food comprising the combination according to claim 1 and auxiliary substances.

12. The product according to claim 11, wherein it is made in the form of a powder, tablet, capsule, bar, snack.

13. The use of the product according to claim 11 for the comprehensive maintenance of the detoxification function and restoration of the liver.
